(19) United States
(12) Patent Application Publication
(10) Pub. No.: US 2010/0173336 A1
(43) Pub. Date: Jul. 8, 2010

(54) METHODS FOR ASSESSMENT OF CARDIOVASCULAR DISEASE RISK

(76) Inventor: Robert L. Stout, Olathe, KS (US)

Correspondence Address: HOVEY WILLIAMS LLP, 10801 Mastin Blvd., Suite 1000, Overland Park, KS, 66210, US

(21) Appl. No.: 12/689,371

(22) Filed: Jan. 19, 2010

Related U.S. Application Data
(63) Continuation of application No. 11/278,651, filed on Apr. 4, 2006, now abandoned.

(51) Int. Cl. C12Q 1/60

(52) U.S. Cl. 435/11

(57) ABSTRACT

Methods are provided for more accurately assessing cardiovascular disease (CVD) risk factors in individuals or populations, using a bimodal analysis including cholesterol-based CVD risk markers together with serum triglyceride levels. Preferably, if a CVD marker (e.g., the ratio of total cholesterol to HDL) yields high risk factors, these factors may be adjusted in inverse relationship to serum triglyceride concentrations. If for example a given marker gives initial risk factors substantially equivalent to relative risk factors of about 1.5 or above, then the initial risk factors can be decreased if serum triglyceride levels are high, or increased if serum triglyceride levels are low. The invention is particularly useful for accurately assigning relative risk of mortality in the life insurance industry, and in decisions about prescribing or withholding medications.

### CROSS-REFERENCE TO RELATED APPLICATION

This is a continuation of identically titled application Ser. No. 11/278,651 filed Apr. 4, 2006, and is incorporated by reference herein in its entirety.

### BACKGROUND OF THE INVENTION

1. Field of the Invention

The present invention is broadly concerned with methods for assessing cardiovascular disease (CVD) risk in individuals and populations by using cholesterol-based CVD risk markers together with serum triglyceride levels as cofactors. More particularly, the invention is concerned with such methods wherein, in appropriate instances, risk factors are adjusted in an inverse relationship to serum triglyceride levels, e.g., where a selected CVD marker gives a relative risk factor of about 1.5 or above, the relative risk factor is adjusted upwardly if the serum triglyceride level is low and is adjusted downwardly if the serum triglyceride level is high.

2. Description of the Prior Art

The Framingham Heart Study investigated the relationship between cardiovascular disease and lipids. The study began in 1948 and continues as the landmark epidemiological milestone of cardiovascular research. The study authors reported a graded relationship between cholesterol and cardiovascular events. Subsequent studies reported a similar relationship with

LDL or low density cholesterol. In contrast, high density cholesterol was reported to have an inverse relationship to risk. The lower the value, the higher the risk. One additional component, serum triglyceride level, was shown to confer a minor increase in risk.

Subsequent markers of CVD risk have included size of LDL particles, biomarkers of inflamation, alteration in coagulation factors, smoking and other environmental factors, and carbohydrate metabolism. While each study has increased our understanding of the factors that contribute to CVD risk, there is still considerable confusion regarding any synergistic relationship between factors which can amplify or reduce risk.

Serum triglyceride levels have been routinely measured as a part of life insurance examinations and risk assessments, but have been thought to be only a weak predictive marker for CVD. In all cases, however, the presence of high serum triglyceride levels above about 200 mg has been thought to increase an individual's risk of CVD, particularly when coupled with high-risk cholesterol-based CVD risk marker values. Thus, the accepted wisdom in the art is that there is a direct proportional relationship between high cholesterol-based CVD risk marker values and high serum triglyceride levels. See, e.g., Griffin et al., Atherosclerosis, 106, 241-53 (1994); Zambon et al., Hyperlipidasmia and Cardiovascular Disease, 9, 329-36 (1998); and

Gardner et al., Jour. Amer. Med. Assn., 276, 875-81 (1996), all of the foregoing being incorporated by reference herein.

### SUMMARY OF THE INVENTION

The present invention provides new techniques for accurately assessing CVD risk factors for individuals and statistically valid populations. The invention is based upon the finding that high levels of serum triglycerides are inversely proportional to cholesterol-based CVD risk marker values indicating high CVD risk. Generally speaking, in one aspect of the invention, methods are provided for assessing CVD risk factors for individuals, comprising the steps of determining at least one cholesterol-based CVD marker for the individuals, as well as the serum triglyceride levels for the individuals. If the markers give CVD risk factors for the individuals substantially equivalent to relative risk factors of about 1.5 or above, these risk factors are adjusted for the individuals in an inverse relationship to the triglyceride levels.

In a related method, a selected CVD measure of association is applied to the CVD marker values in order to determine initial risk factors for the individuals, and such initial factors are then adjusted based upon the serum triglyceride levels. Generally, the risk factors would be adjusted upwardly where serum triglyceride levels are low (e.g., below about 200 mg) and downwardly if the serum triglyceride levels are high (e.g., about 200 mg or above).

The most common CVD measure of association, relative risk, is preferably employed in carrying out the invention. However, other measures of association including odds ratio, absolute risk, and attributable risk can also be used. Typical cholesterol-based CVD risk markers useful in the invention include total cholesterol, HDL (high density cholesterol), LDL, cholesterol particle size distribution, the ratio of total cholesterol (TC) to HDL, the ratio of total cholesterol to LDL, the ratio of HDL to LDL, and combinations thereof; but, the ratio of total cholesterol to HDL is the most preferred marker. When using this preferred marker, the inverse proportional adjustment based upon serum triglyceride level is advantageously employed when the TC:HDL ratio is from about 5 or above.

These methods can also be used for assessing risk factors for a population of individuals, as well as single individuals. When large populations are involved, it is preferred to determine at least one cholesterol-based CVD marker and the serum triglyceride level for all of the individuals in the population. Then, a measure of association is used to assign different initial risk factors to respective individuals in the population, based at least in part upon the value of the markers for respective individuals. These initially assigned risk factors are then adjusted based upon the respective triglyceride levels for the individuals. This adjusting step involves the step of raising the assigned risk factor for respective individuals having low triglyceride levels, and lowering the assigned risk factor for respective individuals having high triglyceride levels.

The invention is particularly applicable for the life insurance industry. Thus, if an individual has a high cholesterol-based CVD risk marker value which would normally cause the individual to be rated by an insurance company to pay high premiums (or be denied insurance altogether), that individual can be more accurately rated using the principles of the invention.

Hence, if the individual has a relatively high serum triglyceride level, the individual would be given a significantly lower rating than would obtain by consideration of the cholesterol-based CVD risk marker values alone. In like manner, an individual having a high CVD risk marker together with a low triglyceride level could be rated higher than under present practices. Indeed, the entire CVD risk rating system of the life insurance industry can be completely reformulated using the methods of the invention, to give insureds proper ratings for their respective conditions.

The invention also finds utility in connection with treatment of patients at risk for CVD. Again, knowledge of the effect of serum triglyceride levels upon CVD risk can be used to either withhold or prescribe medications, or to prescribe other treatment modalities such as increased exercise and diet.

### BRIEF DESCRIPTION OF THE DRAWINGS

FIG. 1 is a graph of relative risk versus the ratio of total cholesterol to HDL for a population of 3,324,569 individuals who applied for life insurance and where 66,722 had died within 12 years after applying for the insurance, and showing the combined effect of total cholesterol to HDL and varying serum triglyceride levels on relative risk for the population;

FIG. 2 is a graph similar to that of FIG. 1, but showing a different grouping of serum triglyceride levels;

FIG. 3 is a graph of relative risk versus the ratio of LDL to HDL for a population of 3,324,569 individuals who applied for life insurance and where 66,722 had died within 12 years after applying for the insurance, and showing the combined effect of LDL to HDL ratios and varying serum triglyceride levels on relative risk for the population;

FIG. 4 is a graph of relative risk versus HDL levels for a population of 3,324,569 individuals who applied for life insurance and where 66,722 had died within 12 years after applying for the insurance, and showing the combined effect of HDL levels and varying serum triglyceride levels on relative risk for the population;

FIG. 5 is a graph of relative risk versus serum triglyceride concentration for a population of 3,324,569 individuals who applied for life insurance and where 66,722 had died within 12 years after applying for the insurance, without regard to any cholesterol-based CVD markers;

FIG. 6 is a graph of LDL particle size distribution versus serum triglyceride levels for a population of 598 individuals 50+ years of age, and demonstrating the metabolic effect of triglyceride levels on the concentrations of least dense and largest particles (A), most dense and smallest particles (B), and particles of intermediate density and size (AB), where “Fraction (%)” refers to the percent of three types of LDL particles in the samples, namely A (largest and least dense), B (smallest and most dense) and AB (intermediate size and density); and FIG. 7 is a graph of Fraction (%) versus LDL:HDL ratio using the data set described in FIG. 6.

### DETAILED DESCRIPTION OF THE PREFERRED EMBODIMENT

Epidemiologists study morbidity and mortality based upon an exposure or explanatory variable, such as any agent, host, or environmental factor that may have an effect upon health, together with the disease in question, a response variable. The goal is to quantify the effect of the exposure variable upon the disease response variable. Generally speaking, this is done by comparing rates of disease in an exposed group versus a non-exposed group.

There are a number of ways of comparing the exposed and non-exposed groups, using different measures of association. A measures of association is any mathematical or statistical measure that used to quantify the association between two or more variables. In the context of epidemiology, a measure of association is any such mathematical or statistical relationship used to measure disease frequency relative to other factors, and is an indication of how more or less likely one is to develop disease as compared to another. Measures of association focus on risk factors which are found to be associated with a health condition, and may be thought of as an attribute or exposure that increases the probability of occurrence of disease (e.g., behavior, genetic, environmental or social factors, time, person or place).

Epidemiological measures of association can broadly be divided into absolute and relative comparisons. Thus, a five-year study of the rate of a disease may yield a rate of 2 per 100 in smokers and 1 per 100 in nonsmokers. An absolute comparison such as (2 per 100)−(1 per 100)=(1 per 100), meaning there is one additional case per 100 smokers. A relative comparison such as (2 per 100)/(1 per 100)=2, means that smokers are at twice the risk of nonsmokers.

A variety of different measures of association have been used in epidemiology. The most common are relative risk RR (also called risk ratio) and odds ratio OR. Risk ratio is often used in cohort studies and may be defined as the relative risk associated with a risk factor, e.g., RR=R1/R0, where R1 is the rate in an exposed group versus R0, the rate in a non-exposed group.

RR is thus a risk multiplier on top of a baseline risk R0, where the segment of the RR above 1 represents elevation in risk. Thus, a RR of 1.0 or greater indicates an increased risk, a RR of less than 1.0 indicates decreased risk, and a RR of 2 represents a 100% increase in risk.

OR is an epidemiological measure of association expressing disease frequency in terms of odds, and is defined as the odds of disease in the exposed population divided by the odds of disease in the unexposed population. OR is more often used in case-controlled studies, and may involve a comparison of disease cases with the prevalence among non-cases for controls. Both RR and OR characterize the association between the exposure and the disease in relative terms, and both reflect the frequency of disease occurrence among exposed subjects as a multiple of the rate among unexposed subjects.

Absolute or difference measures of association are also used in epidemiology, and are generally referred to as attributable risk and population attributable risk percent. Attributable risk is defined as the incidents of disease in an exposed population minus the incidents of disease in the unexposed population, and generally is thought of as the number of cases among the exposed that could be eliminated if the exposure were removed. Population attributable risk percent is defined as the incidents of the disease in the total population minus the incidents in the unexposed population, divided by the incidents of disease in the total population. It measures the excess risk of disease in the total population attributable to exposure and the reduction in risk which would be achieved if the population were entirely unexposed.

Epidemiological measures of association are further defined and explained in: Basic Epidemiology Measures of Association, by Thomas Songer, presented at The South Asian Cardiovascular Research Methodology Workshop and available at www.publichealth.pitt.edu/supercourse/SupercoursePPT/19011-20001/19091.ppt; and Epidemiologic Measures of Association, by Saeed Akhtar and appearing at www.publichealth.pitt.edu/supercourse/SupercoursePPT/80011-9001/8861.ppt. Both of these references are incorporated by reference herein.

In present practice, cholesterol-based markers include total cholesterol, HDL, LDL, cholesterol particle size distribution, the ratio of total cholesterol to LDL, the ratio of HDL to LDL, and combinations thereof. Perhaps the most commonly used marker, particularly in the life insurance industry for rating of insurance applicants in connection with CVD risk, is the ratio of total cholesterol to HDL. The serum triglyceride level is presently used in a directly proportional or additive fashion, i.e., a high serum triglyceride level (e.g., above about 200 mg) leads to a higher assigned risk when coupled with high cholesterol-based CVD risk markers.

The present invention is based upon a newly discovered and counter-intuitive relationship between cholesterol-based CVD risk markers and serum triglyceride levels. As noted above, it has heretofore been thought that high cholesterol-based CVD risk markers, when coupled with high serum triglyceride levels, gave increased CVD risk factors, as compared to risk analyses based only upon the cholesterol-based CVD risk markers. It has now been discovered that the previously held beliefs about the combination of high cholesterol-based CVD risk markers and high triglyceride levels are false. Rather, individuals having high assigned risk factors based upon cholesterol-based CVD risk markers, together with high serum triglyceride levels, actually have significantly lower risks of CVD. Moreover, individuals having high assigned risk factors based upon cholesterol-based CVD risk markers, coupled with low serum triglyceride levels, have higher CVD risks than are predicted by analyses of the cholesterol-based CVD risk markers alone. Thus, there exists a wholly unexpected inverse relationship between triglyceride levels and cholesterol-based CVD risk marker levels.

The foregoing relationships are confirmed by the study of a population of 3,324,569 insurance applicant samples which were analyzed for cholesterol-based CVD markers and serum triglyceride levels. The study involved a twelve-year period, over which time 66,722 of the applicants had died after applying for insurance. The cholesterol-based CVD markers were compared between the two populations, and relative mortality risks were calculated.

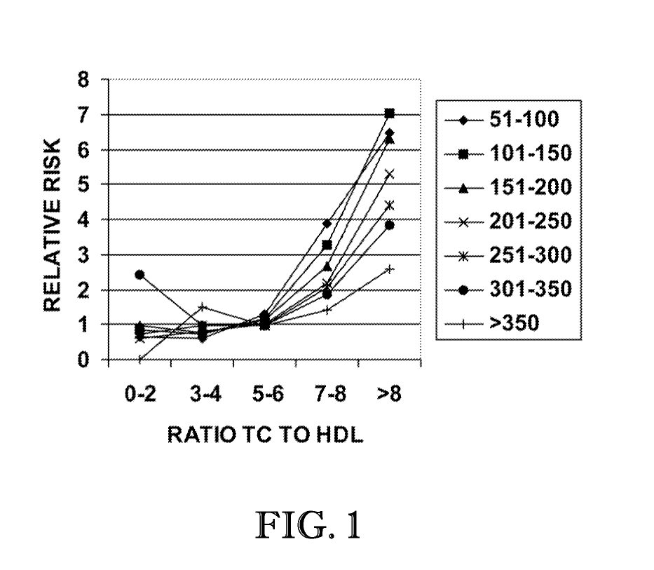

Referring first to FIG. 1, a graph of relative risk versus the ratio of total cholesterol to HDL is presented, showing the effect of various levels of serum triglyceride. Note that the relative risks for individuals having a total cholesterol:HDL ratio of about 5 and above are inversely proportional to the serum triglyceride levels. Thus, for individuals having the highest serum triglyceride levels greater than 350 mg, the relative risks are significantly lower than individuals having lower serum triglyceride levels of less than about 300 mg. The effect is particularly pronounced with the lowest serum triglyceride levels of less than about 200 mg. Also, at above about 1.5 relative risk, the inverse triglycerides effect becomes most pronounced.

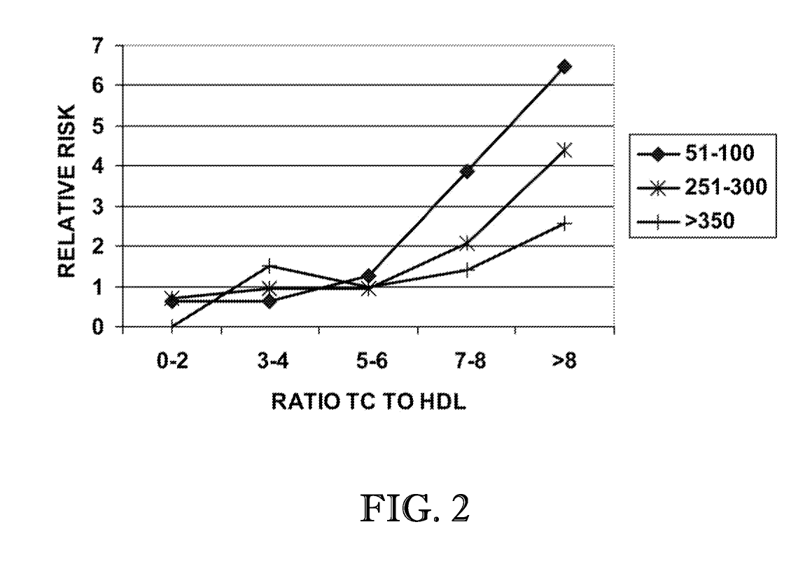

FIG. 2 is similar to FIG. 1, but shows a different grouping of serum triglyceride levels, namely below about 100 mg, between about 250 and 300 mg, and greater than 350 mg. Here again, the inverse effect of serum triglyceride levels is evident, with the highest serum triglyceride levels having risk factors well below the lower serum triglyceride levels.

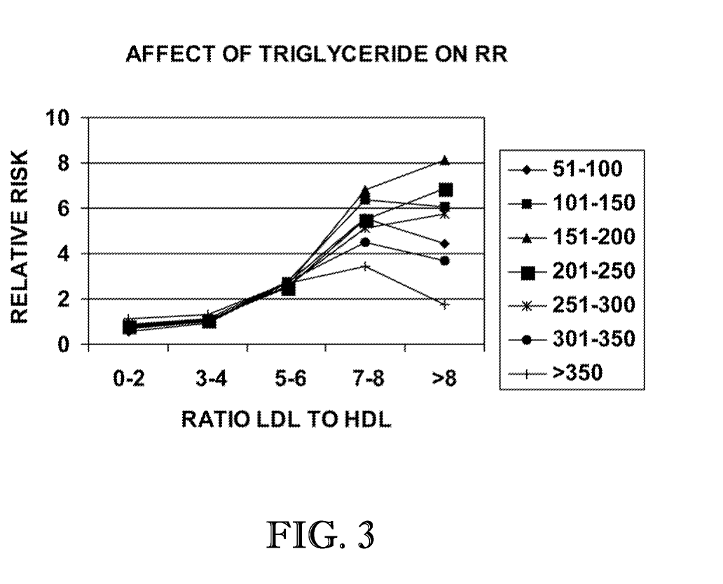

This effect is substantially consistent throughout various types of cholesterol-based CVD risk markers. Referring to FIG. 3, a graph of relative risks versus LDL:HDL ratios, the effect of varying serum triglyceride levels is shown. Above LDL:HDL ratios of about 5, individuals having the highest serum triglyceride levels have the lowest relative risk. In this data, the triglyceride effect is most pronounced at triglyceride levels at above about 300.

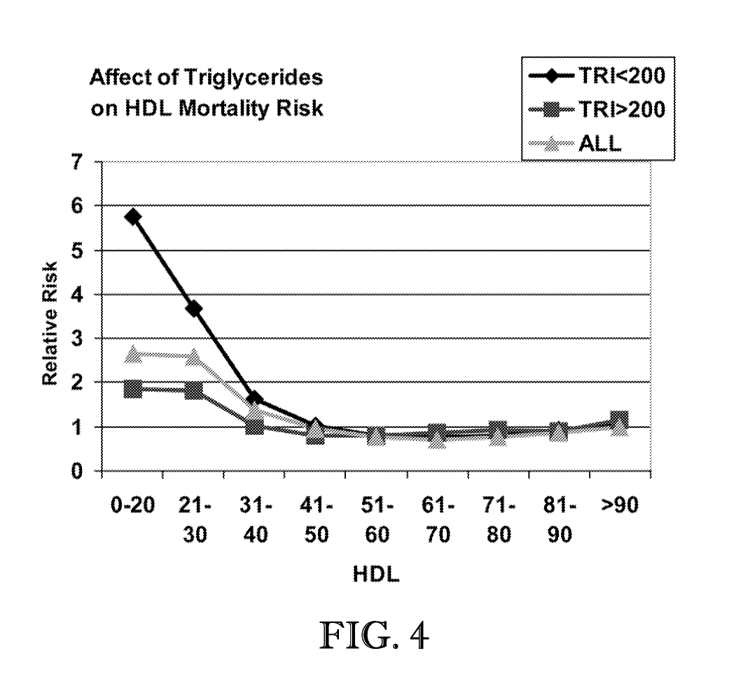

FIG. 4 illustrates the same relationship, where individuals having an HDL level of below about 40 had low relative risks if their serum triglyceride levels were above 200, and had high relative risks if their serum triglyceride levels were below 200. It will further be observed that at relative risk levels of about 1.5 and above, the inverse triglyceride effect becomes most evident.

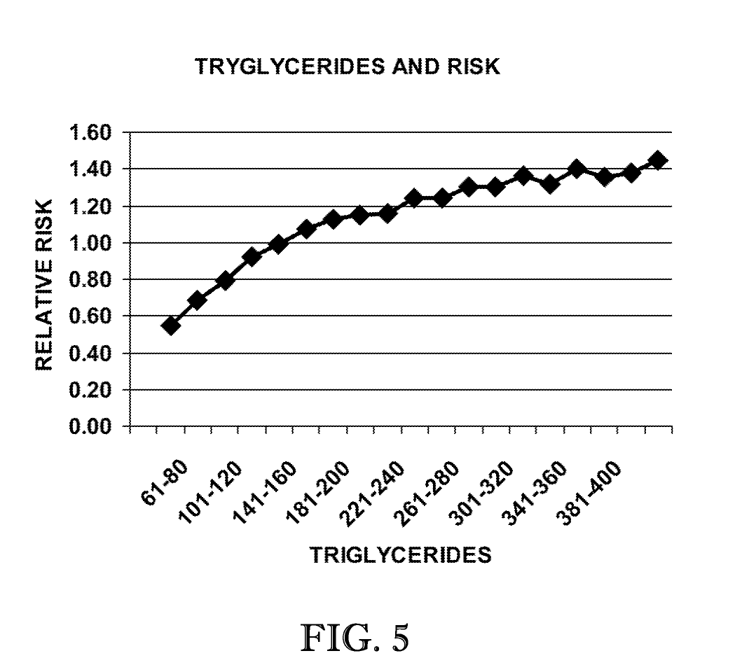

There is apparently a heretofore unappreciated in vivo metabolic relationship between serum triglyceride levels and cholesterol species and concentrations, which affect assigned CVD risk factors, and which possibly explains the results graphically depicted in FIGS. 1-4. FIG. 5 demonstrates that higher serum triglyceride levels alone, without consideration of cholesterol-based CVD risk markers, gives a direct proportional relationship between relative risk and serum triglyceride levels. Thus, as the serum triglyceride levels increase, relative risks increase. Hence, the presence of high serum triglyceride levels alone cannot explain the results of FIGS. 1-4.

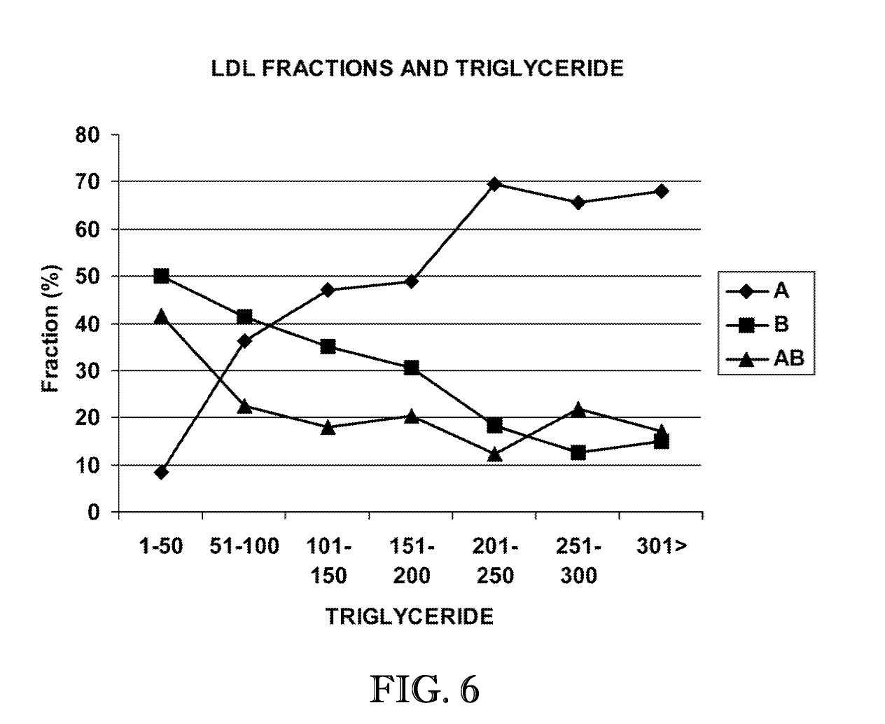

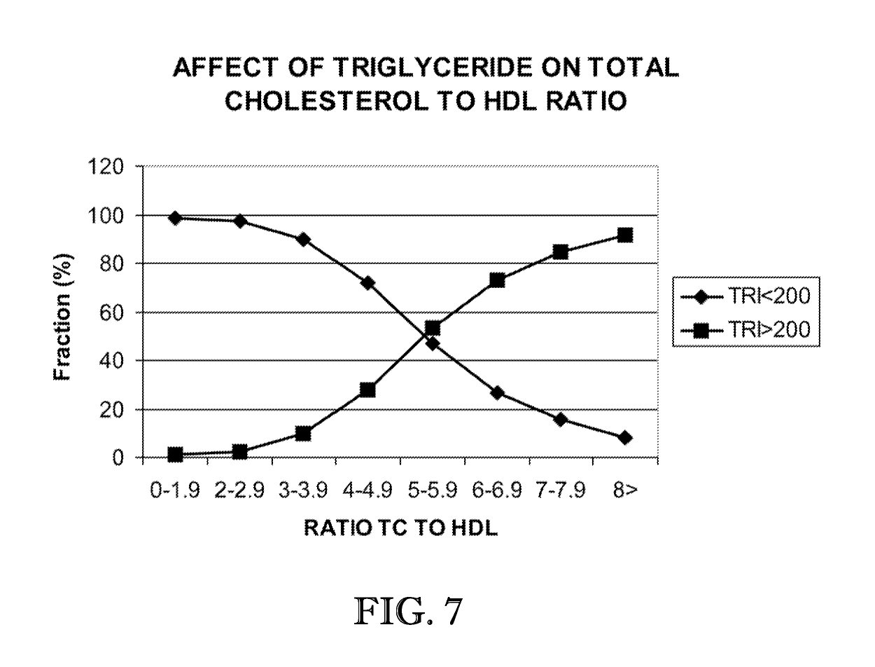

However, FIG. 6 illustrates that higher serum triglyceride levels lead to a significant lowering in the smallest and most dense LDL particles, considered to be the most likely to increase CVD risk. That is, the smaller LDL cholesterol particles, owing to their increased densities, are believed to more readily agglomerate in the arteries, leading to CVD. Accordingly, any lowering of the LDL cholesterol fraction is deemed to be beneficial. Thus, individuals having high serum triglyceride levels of above 200 mg, the concentration of small, dense LDL and intermediate size and density particles is significantly reduced. This may further explain the inverse triglyceride phenomenon of the present invention.

FIG. 7 is similar and shows the effect of high and low serum triglyceride levels on LDL:HDL ratios. For example, with the highest LDL:HDL ratios of 8 and above, the presence of serum triglyceride levels above 200 mg gives a substantial reduction in the small, low density particles. On the other hand, with triglyceride levels below 200 mg, the fraction of low density particles is reversed. In the case of low total cholesterol/HDL ratios, high triglyceride levels have the opposite effect, namely there is no reduction in low density particles, whereas at triglyceride levels greater than 200 mg, the lowering of LDL particles is manifest.

The results exemplified in FIGS. 1-4 were based upon a relative risk measure of association, showing that the effect of serum triglyceride levels begins to become evident at relative risk factors of about 1.5 and above, and especially at about 2 and above. However, the invention is not limited to the use of relative risk as a measure of association, in that a variety of different measures of association may be employed, such as those discussed previously. Use of different measures of association will typically give different threshold values for the application of the serum triglyceride level-based risk factor adjustments of the invention. As such, it would be difficult or impossible to assign threshold values for all of the different measures of association which may be employed. As a general proposition though, a selected and valid cholesterol-based CVD risk marker will give values which are substantially equivalent to those obtained in a relative risk analysis, i.e., the different measures of association values are either substantially identical with the corresponding relative risk values, or are mathematically or statistically related to such relative risk values. Thus, if a selected cholesterol-based CVD risk marker gives a risk factor which is substantially equivalent to a relative risk factor of about 1.5 or above, then the risk factor adjustments of the invention using serum triglyceride levels can be employed.

It will also be appreciated that the general inverse relationship between triglyceride levels and typical cholesterol based cholesterol-based CVD risk markers can be expressed in a number of ways. This can be done by way of a lookup table, i.e., a given CVD risk marker value and a serum triglyceride level are inputted, and an adjusted or final risk factor is assigned. Alternately, the relationship may be expressed as ratios of CVD risk marker values and serum triglyceride levels, or algorithms may be devised expressing appropriate relationships between CVD risk markers and serum triglyceride levels. The present invention embraces all such alternatives.

I claim:

1. In a method of assessing a cardiovascular disease (CVD) risk factor for an individual, including the steps of determining at least one cholesterol-based CVD marker for the individual, and the serum triglyceride level for the individual, and wherein the individual has a CVD risk factor substantially equivalent to a relative risk factor of about 1.5 or above, the improvement which comprises adjusting the individual's risk factor in an inverse relationship to said triglyceride level.

2. The method of claim 1, said relative risk factor being about 2 or above.

3. The method of claim 1, including the steps of applying a selected CVD measure of association to the CVD marker, determining an initial risk factor for the individual based at least in part upon said CVD marker, and adjusting the initial risk factor based upon said serum triglyceride level.

4. The method of claim 3, said measure of association selected from the group consisting of relative risk, odds ratio, absolute risk, and attributable risk.

5. The method of claim 4, said measure of association being relative risk.

6. The method of claim 1, including the step of lowering said risk factor if said triglyceride level is above about 200, and raising said risk factor if said triglyceride level is below about 200.

7. The method of claim 1, said cholesterol-based CVD marker selected from the group consisting of total cholesterol, HDL, LDL, cholesterol particle size distribution, the ratio of total cholesterol to HDL, the ratio of total cholesterol to LDL, the ratio of HDL to LDL, and combinations thereof.

8. The method of claim 7, said marker being the ratio of total cholesterol to HDL.

9. The method of claim 8, including the step of adjusting said risk factor if the individual's ratio of total cholesterol to HDL is from about 5 or above.

10. The method of claim 1, including the step of adjusting the individual's life insurance rating based upon said adjusted assessment of risk of CVD.

11. The method of claim 1, including the step of withholding or prescribing medication based upon said adjusted assessment of risk of CVD.

12. A method of assessing cardiovascular disease (CVD) risk factors for a population of individuals, comprising the steps of:
determining at least one cholesterol-based CVD marker for all of the individuals in said population, and the serum triglyceride levels for all of the individuals in said population;
using a measure of association to assign different risk factors to respective individuals in said population, based at least in part upon the markers for respective individuals; and
adjusting the assigned risk factor for respective individuals in said population, based upon the respective triglyceride levels for the individuals,
said adjusting step comprising the step of raising the assigned risk factor for respective individuals having low triglyceride levels, and lowering the assigned risk factor for respective individuals having high triglyceride levels.

13. The method of claim 12, said measure of association selected from the group consisting of relative risk, odds ratio, absolute risk, and attributable risk.

14. The method of claim 13, said measure of association being relative risk.

15. The method of claim 12, including the step of carrying out said adjusting step for individuals having an assigned risk factor substantially equivalent to a relative risk factor of from about 1.5 or above.

16. The method of claim 15, said relative risk factor being about 2 or above.

17. The method of claim 12, including the step of lowering said risk factor if said triglyceride level is above about 200, and raising said risk factor if said triglyceride level is below about 200.

18. The method of claim 12, said cholesterol-based CVD marker selected from the group consisting of total cholesterol, HDL, LDL, cholesterol particle size distribution, the ratio of total cholesterol to HDL, the ratio of total cholesterol to LDL, the ratio of HDL to LDL, and combinations thereof.

19. The method of claim 18, said marker being the ratio of total cholesterol to HDL.

20. The method of claim 19, including the step of adjusting said risk factor if the individual's ratio of total cholesterol to HDL is from about 5 or above.

21. The method of claim 12, including the step of adjusting the life insurance rating of respective individuals in said population, based upon said adjusted assessment of risk of CVD.
